(19) United States
(12) Patent Application Publication
(10) Pub. No.: US 2026/0105985 A1
(43) Pub. Date: Apr. 16, 2026

(54) METHODS OF VIRAL PROBE DESIGN

(71) Applicant: ILLUMINA, INC., San Diego, CA (US)

(72) Inventors: Keith Arora-Williams, Los Angeles, CA (US); Kate Broadbent, Salt Lake City, UT (US)

(21) Appl. No.: 19/422,743

(22) Filed: Dec. 17, 2025

Related U.S. Application Data
(63) Continuation of application No. PCT/US2024/049837, filed on Oct. 3, 2024.
(60) Provisional application No. 63/588,345, filed on Oct. 6, 2023.

(51) Int. Cl. G16B 25/20; G16B 20/50; G16B 40/30

(52) U.S. Cl. CPC G16B 25/20; G16B 20/50; G16B 40/30

(57) ABSTRACT

The disclosed embodiments concern methods for designing probes for improving environmental sample (including wastewater samples and other samples) surveillance and surveillance of other samples for various viruses. In certain embodiments described herein, methods and systems are provided for designing a pool of probes for enriching a sample for one or more target viral nucleic acids.

### CROSS-REFERENCE TO RELATED APPLICATION

This application is continuation claiming priority to PCT/2024/049837, filed Oct. 3, 2024, which claims the benefit of priority of U.S. Provisional Application No. 63/588,345, filed Oct. 6, 2023, the contents of which are each incorporated by reference herein in their entireties for any purpose.

### DESCRIPTION

### Technical Field

This disclosure relates to methods of designing probes to enrich for various viruses from complex sample types that contain few viral particles, such as wastewater, or from other samples collected from the environment, laboratory, or of biological origin. Libraries enriched with the present methods may be used to generate sequencing data.

### Background

Viruses continue to develop naturally resulting in new strains and diseases to human populations. For example, the World Health Organization (WHO) declared infection by the novel Severe Acute Respiratory Syndrome Coronavirus 2 (SARS-CoV-2) as a pandemic and termed the related disease as coronavirus disease 2019 (COVID-19). SARS-CoV-2 can be detected in feces. Additionally, most persons infected with enterically transmitted viruses shed large amounts of virus in feces for days or weeks, both before and after onset of symptoms. Therefore, viruses causing gastroenteritis may be detected in wastewater, even if only a few persons are infected. The abundance and diversity of pathogenic viruses in wastewater has been shown to reflect the pattern of infection in human population. Adenovirus, rotavirus, hepatitis A virus, and other enteric viruses, such as norovirus, coxsackievirus, echovirus, reovirus and astrovirus are some of the principal human pathogenic viruses transmissible via water media.

Viruses are ubiquitous and persistent in raw wastewater and treated wastewater. One of the main sources of viruses, including viral pathogens in wastewater is human fecal matter, particularly that from infected persons. Sewage systems receive enteric viruses excreted by infected individuals. In addition to human pathogenic viruses, waterborne viruses that originate from food production, animal husbandry, seasonal surface runoff and other sources are present in wastewater. Wastewater can serve as a significant source of information for public health and agricultural officials on the pathogens present in a population and the levels of those pathogens.

The bodies that receive treated wastewater are oftentimes used for recreational activities and agriculture, and as a source of raw water for drinking water production. The presence of potentially pathogenic viruses in wastewater is of concern since it can pose risks to human health. While this presents an opportunity to investigate wastewater for incidence of disease or presence of potentially pathogenic viruses, sampling and measuring wastewater for a virus-of-interest is problematic due to low concentrations of this virus or particles thereof alone. The mixture of contaminants (e.g., other waterborne pathogens including bacterial, fungal, and parasitic pathogens, as well as viruses not of interest or human nucleic acids) and a virus-of-interest presents a difficult medium for viral DNA and RNA extraction therefrom, especially where concentrations of a virus-of-interest are low. As such, methods of enriching wastewater samples for viral targets are needed to quantify incidence of viral infection or disease in a community and to identify novel viruses of interest in wastewater, such as from a sewer system, and methods of recovering nucleic acids from a virus-of-interest in wastewater.

Described herein are methods to design viral probes for enrichment and detection of novel strains or variants of genetically related viruses of interest. Through an iterative design process, the viral probes are optimized to capture a broad diversity of viral sequences to increase the chance of capturing genomic sequence from a yet to be discovered strain or novel variant coronavirus or other virus-of-interest. The viral probe design methods described herein also minimize probe redundancy to reduce the overall number of oligonucleotides that are necessary to detect such a broad diversity of viral sequences.

### SUMMARY

In accordance with the description, described herein are methods of viral probe design for one or more virus-of-interest nucleic acids and/or for improving environmental sample surveillance for various viruses. It should be understood that the methods and processes discussed herein may include:

Embodiment 1. An iterative method of designing a pool of probes for enriching a sample for one or more target viral nucleic acids comprising the steps of: (a) optionally clustering a plurality of reference sequences to produce a plurality of clusters; (b) designing a plurality of tiled probes based on the clusters produced in step (a) or on another subset of total reference sequences that has been selected using another approach such as longest reference sequence per taxonomic identifier, wherein a desired probe length and gap length between probes is specified; (c) clustering the plurality of tiled probes designed in step (b) with a threshold identity (e.g., 75%, 85%, 90%, 95%, 99% or 100% identity); (d) optionally replacing ambiguous bases within the tiled probes based on a threshold tolerance; (e) mapping the probes to all reference sequences and gap-filling based on a mismatch tolerance (mapping and gap-filling can be iterated up to n rounds, where n=the total # of available reference sequences per virus); (f) optionally scoring the plurality of probes and predicting problematic probes; (g) optionally performing one or both of removing problematic probe sequences or adding backup probes to supplement problematic probes; (h) optionally further predicting difficult to synthesize probes; (i) optionally replacing difficult to synthesize probes predicted in step (h) with a mutated probe; and (j) optionally replacing ambiguous bases in the backup and/or mutated probes based on the threshold tolerance in step (d) or a different threshold tolerance.

Embodiment 2. The method of embodiment 1, wherein the reference sequence clustering step (a) is carried out with a threshold identity (e.g., 75%, 85%, 90%, 95%, 99% or 100% identity).

Embodiment 3. The method of embodiment 1 or 2, wherein problematic probes are predicted using one or more base metric rules.

Embodiment 4. The method of any one of embodiments 1-3, wherein problematic probes are predicted using GC content, homopolymer length, and/or Shannon entropy calculations.

Embodiment 5. The method of any one of embodiments 1-4, further comprising scoring the best backup probes to supplement problematic probes.

Embodiment 6. The method of any one of embodiments 1-5, wherein a desired gap length (e.g., 0 bp, 50 bp, 80 bp, 100 bp, 150 bp, 200 bp, 500 bp, 1000 bp, and so forth) between probes (e.g., probes having a length of approximately 80-mer to approximately 120-mer) is specified.

Embodiment 7. The method of any one of embodiments 1-6, wherein the probes have a 0%, 1%, 5%, 10%, 15%, 20%, 25%, or 30% mismatch tolerance.

Embodiment 8. The method of any one of embodiments 1-7, wherein ambiguous bases are replaced with a 1% to 10% tolerance.

Embodiment 9. The method of any one of embodiments 1-8, wherein the problematic probe sequences are determined by cross hybridization, human homology checks, ribosomal operon homology checks, transposable element checks, and/or by identifying low-complexity regions of target sequences.

Embodiment 10. The method of any one of embodiments 1-9, wherein the mutated probe has from 1 to 10 mutations.

Embodiment 11. The method of any one of embodiments 1-10, further comprising producing the pool of probes.

Embodiment 12. The method of any one of embodiments 1-11, wherein the threshold identity for probe clustering comprises identity in the range of 75% to 100% (e.g., 75%, 85%, 90%, 95%, 99% or 100% identity).

Embodiment 13. The method of any one of embodiments 1-12 implemented as an interval design process, wherein all possible probes per target are designed and scored based on, but not limited to, GC content, homopolymer length, and/or Shannon entropy calculations. In further aspects an optimal set of non-overlapping probes is selected using an interval scheduling technique.

Embodiment 14. The method of any one of embodiments 1-13 implemented with probe overlap optimization to reduce probe redundancy, wherein the process iterates over every unique group of overlapping probes (pairs and trios) until the collapse rate plateaus.

Embodiment 15. A probe design system comprising: a processor configured to execute processor-executable routines and a tangible memory medium storing processor executable routines. The processor-executable routines, when executed by the processor, cause actions to be performed comprising: optionally clustering a plurality of reference sequences to produce a plurality of clusters; designing a plurality of tiled probes based on the clusters or on another subset of total reference sequences that has been selected using another approach such as longest reference sequence per taxonomic identifier, wherein a desired probe length and gap length between probes is specified; clustering the plurality of tiled probes with a threshold identity (e.g., 75%, 85%, 90%, 95%, 99% or 100% identity); optionally replacing ambiguous bases within the tiled probes based on a threshold tolerance; mapping the probes to all reference sequences and gap-filling based on a mismatch tolerance (mapping and gap-filling can be iterated up to n rounds, where n=the total # of available reference sequences per virus); optionally scoring the plurality of probes and predicting problematic probes; optionally performing one or both of removing problematic probe sequences or adding backup probes to supplement problematic probes; optionally further predicting difficult to synthesize probes; optionally replacing difficult to synthesize probes with a mutated probe; and optionally replacing ambiguous bases in the backup and/or mutated probes based on the threshold tolerance or a different threshold tolerance.

Embodiment 16. The probe design system of embodiment 15, wherein the reference sequence clustering step (a) is carried out with a threshold identity (e.g., 75%, 85%, 90%, 95%, 99% or 100% identity).

Embodiment 17. The probe design system of embodiment 15 or 16, wherein problematic probes are predicted using one or more base metric rules.

Embodiment 18. The probe design system of any one of embodiments 15-17, wherein problematic probes are predicted using GC content, homopolymer length, and/or Shannon entropy calculations

Embodiment 19. The probe design system of any one of embodiments 15-18 further comprising scoring the best backup probes to supplement problematic probes.

Embodiment 20. The probe design system of any one of embodiments 15-19, wherein a desired gap length (e.g., 0 bp, 50 bp, 80 bp, 100 bp, 150 bp, 200 bp, 500 bp, 1000 bp, and so forth) between probes (e.g., probes having a length of approximately 80-mer to approximately 120-mer) is specified.

Embodiment 21. The probe design system of any one of embodiments 15-20, wherein the probes have a 0%, 1%, 5%, 10%, 15%, 20%, 25%, or 30% mismatch tolerance.

Embodiment 33. The probe design system of any one of embodiments 15-21, wherein ambiguous bases are replaced with a 1% to 10% tolerance.

Embodiment 23. The probe design system of any one of embodiments 15-22, wherein the problematic probe sequences are determined by cross hybridization, human homology checks, ribosomal operon homology checks, transposable element checks, and/or by identifying low-complexity regions of target sequences.

Embodiment 24. The probe design system of any one of embodiments 15-23, wherein the mutated probe has from 1 to 10 mutations.

Embodiment 25. The probe design system of any one of embodiments 15-24, further causing production of the pool of probes.

Embodiment 26. The probe design system of any one of embodiments 15-25, wherein the threshold identity for probe clustering comprises identity in the range of 75% to 100% (e.g., 75%, 85%, 90%, 95%, 99% or 100% identity).

Embodiment 27. The probe design system of any one of embodiments 15-26, implemented as an interval design process, wherein all possible probes per target are designed and scored based on, but not limited to, GC content, homopolymer length, and/or Shannon entropy calculations. In further aspects an optimal set of non-overlapping probes is selected using an interval scheduling technique.

Embodiment 28. The probe design system of any one of embodiments 15-27, implemented with probe overlap optimization to reduce probe redundancy, wherein the process iterates over every unique group of overlapping probes (pairs and trios) until the collapse rate plateaus.

Embodiment 29. One or more tangible computer-readable media encoding processor-executable routines, wherein the processor-executable routines comprise: code for optionally clustering a plurality of reference sequences to produce a plurality of clusters; code for designing a plurality of tiled probes based on the clusters or on another subset of total reference sequences that has been selected using another approach such as longest reference sequence per taxonomic identifier, wherein a desired probe length and gap length between probes is specified; code for clustering the plurality of tiled probes with a threshold identity (e.g., 75%, 85%, 90%, 95%, 99% or 100% identity); code for optionally replacing ambiguous bases within the tiled probes based on a threshold tolerance; code for mapping the probes to all reference sequences and gap-filling based on a mismatch tolerance (mapping and gap-filling can be iterated up to n rounds, where n=the total # of available reference sequences per virus); code for optionally scoring the plurality of probes and predicting problematic probes; code for optionally performing one or both of removing problematic probe sequences or adding backup probes to supplement problematic probes; code for optionally further predicting difficult to synthesize probes; code for optionally replacing difficult to synthesize probes with a mutated probe; and code for optionally replacing ambiguous bases in the backup and/or mutated probes based on the threshold tolerance or a different threshold tolerance.

Embodiment 30. The one or more tangible computer-readable media of embodiment 29, wherein the reference sequence clustering step (a) is carried out with a threshold identity (e.g., 75%, 85%, 90%, 95%, 99% or 100% identity).

Embodiment 31. The one or more tangible computer-readable media of embodiment 29 or 30, wherein problematic probes are predicted using one or more base metric rules.

Embodiment 32. The one or more tangible computer-readable media of embodiments 29-31, wherein problematic probes are predicted using GC content, homopolymer length, and/or Shannon entropy calculations

Embodiment 33. The one or more tangible computer-readable media of any one of embodiments 29-32 further comprising scoring the best backup probes to supplement problematic probes.

Embodiment 34. The one or more tangible computer-readable media of any one of embodiments 29-33, wherein a desired gap length (e.g., 0 bp, 50 bp, 80 bp, 100 bp, 150 bp, 200 bp, 500 bp, 1000 bp, and so forth) between probes (e.g., probes having a length of approximately 80-mer to approximately 120-mer) is specified.

Embodiment 35. The one or more tangible computer-readable media of any one of embodiments 29-34, wherein the probes have a 0%, 1%, 5%, 10%, 15%, 20%, 25%, or 30% mismatch tolerance.

Embodiment 36. The one or more tangible computer-readable media of any one of embodiments 29-35, wherein ambiguous bases are replaced with a 1% to 10% tolerance.

Embodiment 37. The one or more tangible computer-readable media of any one of embodiments 29-36, wherein the problematic probe sequences are determined by cross hybridization, human homology checks, ribosomal operon homology checks, transposable element checks, and/or by identifying low-complexity regions of target sequences.

Embodiment 38. The one or more tangible computer-readable media of any one of embodiments 29-37, wherein the mutated probe has from 1 to 10 mutations.

Embodiment 39. The one or more tangible computer-readable media of any one of embodiments 29-38, further causing production of the pool of probes.

Embodiment 40. The one or more tangible computer-readable media of any one of embodiments 29-39, wherein the threshold identity for probe clustering comprises identity in the range of 75% to 100% (e.g., 75%, 85%, 90%, 95%, 99% or 100% identity).

Embodiment 41. The one or more tangible computer-readable media of any one of embodiments 29-40, implemented as an interval design process, wherein all possible probes per target are designed and scored based on, but not limited to, GC content, homopolymer length, and/or Shannon entropy calculations. In further aspects an optimal set of non-overlapping probes is selected using an interval scheduling technique.

Embodiment 42. The one or more tangible computer-readable media of any one of embodiments 29-41, implemented with probe overlap optimization to reduce probe redundancy, wherein the process iterates over every unique group of overlapping probes (pairs and trios) until the collapse rate plateaus.

### BRIEF DESCRIPTION OF THE FIGURES

FIG. 1 shows a multistep methodology for comprehensive and curated sourcing of viral reference sequences to develop viral probes. Viral reference sequence sourcing relies on both publicly available data, proprietary in-house resources, and subject matter expertise (SME) to ensure appropriate configuration. With respect to the in-house metadata filter, for example, steps may be performed that include, but are not limited to: (1) filtering out sequences based on poor quality (e.g., exclude “unverified” sequences) and/or (2) filtering out zoonotic sequences with precision (e.g., retain norovirus sequences isolated from shellfish but exclude canine and feline norovirus sequences).

FIG. 2 shows an example of non-redundant viral genome counts sourced by two methods: In-house, SME-driven comprehensive and curated viral reference sequence sourcing (middle) versus a naïve ˜1 Refseq genome per virus approach (lower right). In the depicted example, there is 100% clustering of source sequences and the viral genome count for segmented viruses=#sequences/#expected segments.

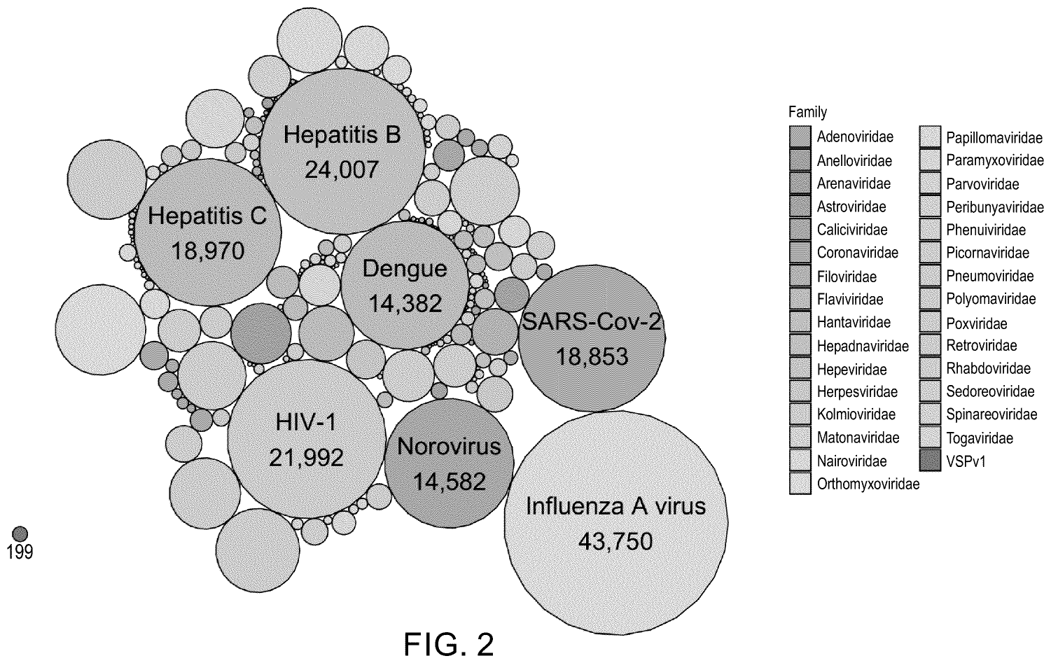

FIG. 3 provides phylogenetic trees depicting the impact on panel design: (1) naïve vs. (2) SME-driven viral genome sourcing for Lassa virus.

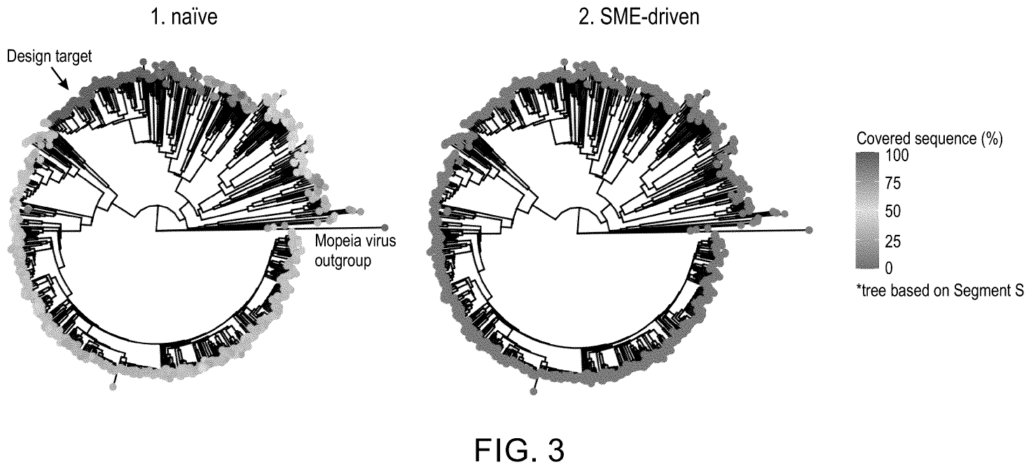

FIG. 4 provides phylogenetic trees depicting the impact on panel design: (1) naïve vs. (2) SME-driven viral genome sourcing for HIV-1.

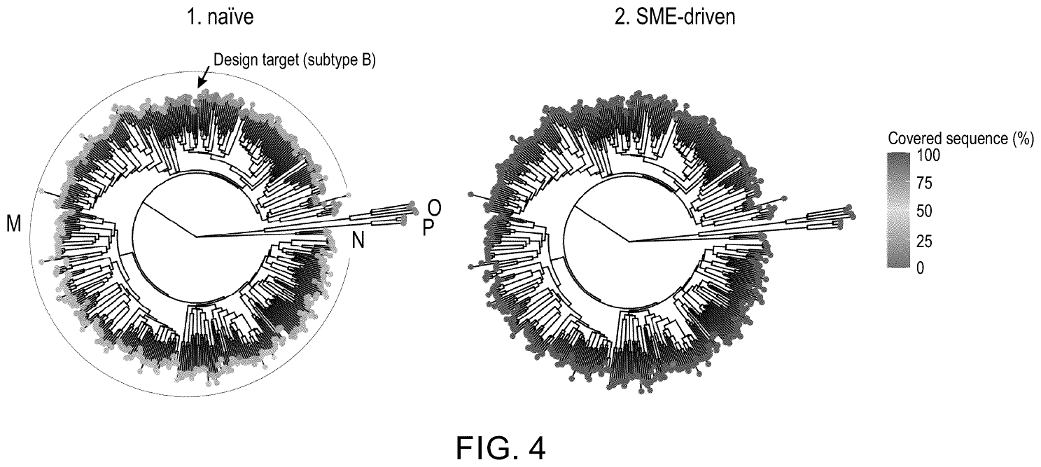

FIG. 5 provides phylogenetic trees depicting the impact on panel design: (1) naïve vs. (2) SME-driven viral genome sourcing for HIV-2.

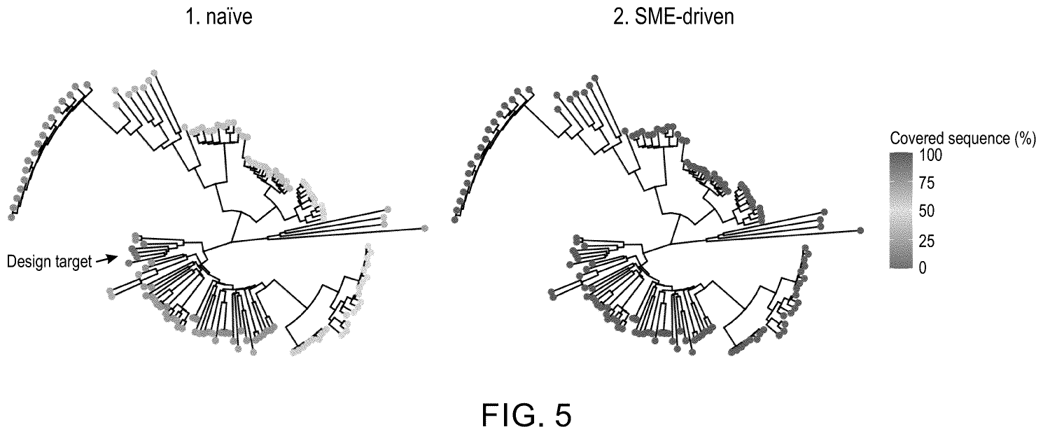

FIG. 6 shows a detailed iterative viral probe design pipeline with two rounds of reference selection and gap-filling. The process is performed per viral target.

FIG. 7 shows a simplified iterative viral probe design pipeline with two rounds of reference selection and gap-filling. The process is performed per viral target. For diverse viruses, rather than design probes on all available reference sequences (or the 95% clustered representative reference sequences), at least two rounds of iterative reference selection and probe design per virus effectively reduces probe count by up to 90%. It should be noted that the first round of reference selection greatly impacts probe count, and the optimal selection strategy may differ per virus. Therefore, trying multiple first round reference selection strategies in parallel is useful to identify the optimal strategy per virus. The iterative design process shown may be implemented for up to n rounds of reference selection and gap filling, where n=the total # of available reference sequences per virus.

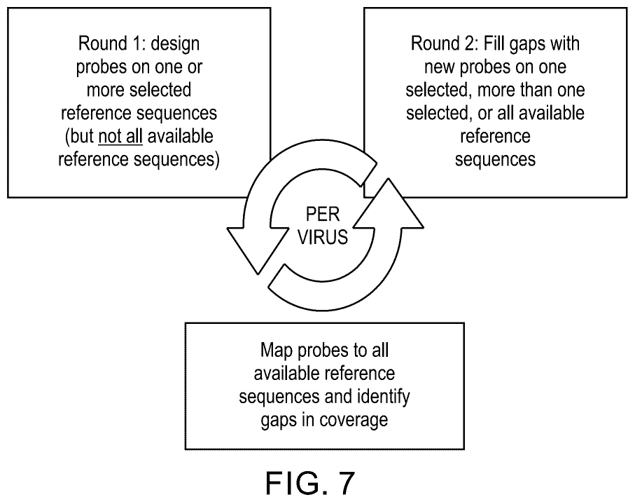

FIG. 8 shows an example of using the iterative probe design pipeline with norovirus as a moderate viral genome diversity example. In this example, total non-redundant norovirus reference sequences sourced for viral genome probe design were 3,026. The total 95% clustered representative norovirus reference sequences were 234. Using an iterative reference selection plus gap-filling approach reduced probe count by more than 50%. In this example, a set of Refseq reference genome sequences, each from a different human norovirus genotype, was selected for Round 1 probe design.

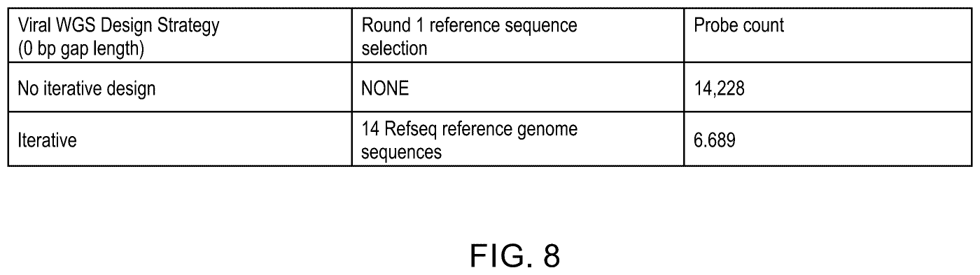

FIG. 9 shows an example of using the iterative probe design pipeline with HIV-1. In this example, total non-redundant HIV-1 reference sequences sourced for viral genome probe design were 23,734. The total 95% clustered representative HIV-1 reference sequences were 15,318. Using an iterative reference selection plus gap-filling approach reduced probe count by more than 90%. In this example, the optimal Round 1 reference selection identified was a set of reference genome sequences representing the longest sequence from all HIV-1 taxonomic identifiers (NCBI taxids).

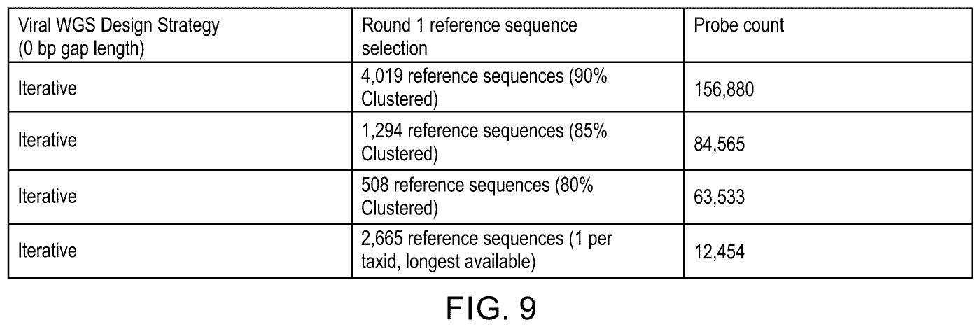

FIG. 10 shows an interval viral probe design technique. In accordance with one such implementation, all possible probes per target are designed and scored (top). Probe scoring may be based on, but is not limited to, GC content, homopolymer length, and/or Shannon entropy calculations. An optimal set of non-overlapping probes is selected using an interval scheduling executable routine (bottom).

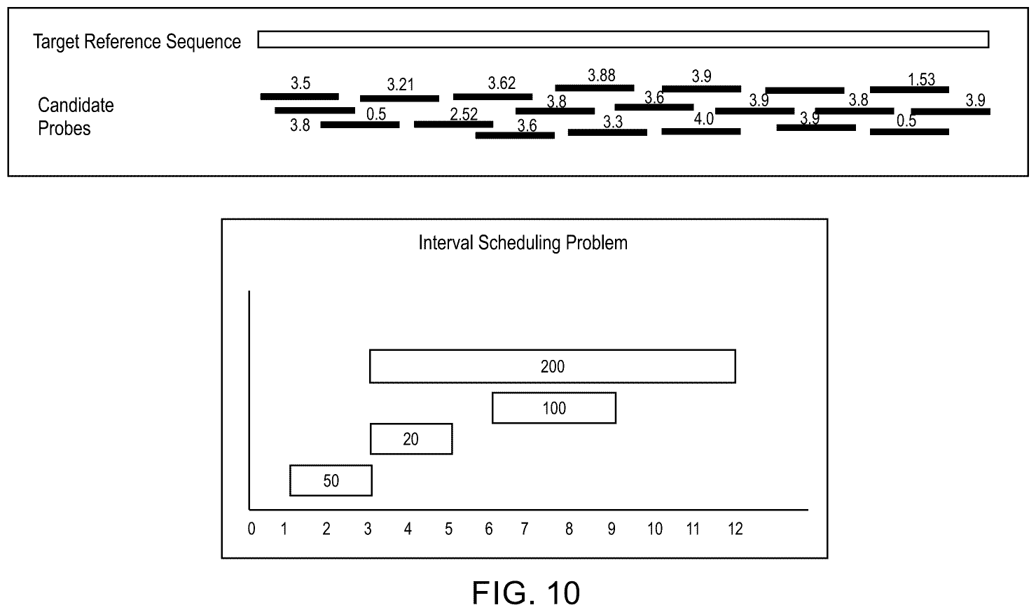

FIG. 11 shows a probe overlap optimization technique in which the process iterates over every unique group of overlapping probes (pairs and trios) until collapse rate plateaus.

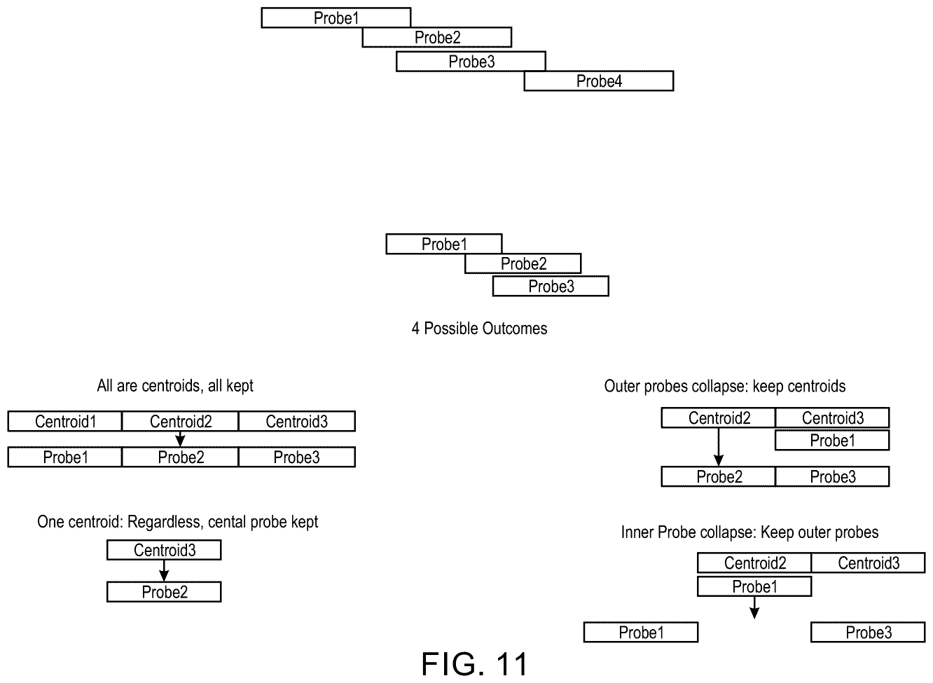

### DESCRIPTION OF THE EMBODIMENTS

The following detailed description of certain examples will be better understood when read in conjunction with the appended drawings. To the extent that the figures illustrate diagrams of the functional blocks of various examples, the functional blocks are not necessarily indicative of the division between hardware components. For example, one or more of the functional blocks (e.g., processors or memories) may be implemented in a single piece of hardware (e.g., a general-purpose signal processor or random-access memory, hard disk, or the like). Similarly, the programs may be stand-alone programs, may be incorporated as subroutines in an operating system, may be functions in an installed software package, and the like. The various examples are not limited to the arrangements and instrumentality shown in the drawings.

### Methods of Designing Probe Sequences

Described herein are methods of designing a pool of probes for enriching a sample for one or more target viral nucleic acids comprising the steps of: (a) clustering a plurality of reference sequences; (b) designing a plurality of tiled probes based on the clusters produced in step (a), wherein desired gap length between probes is specified; (c) clustering the plurality of probes designed in step (b) with a threshold (e.g., 75%, 85%, 90% 95%, 99%, or 100%) identity; (d) replacing ambiguous bases; (e) mapping the probes to all reference sequences and gap-filling; (f) scoring the plurality of probes and predicting problematic probes; (g) removing problematic probe sequences; (h) predicting problematic probes using a machine learning (ML) model; (i) replacing each of the problematic probes predicted in step (h) with a mutated probe; and (j) replacing ambiguous bases.

Further described herein are probe design systems comprising: a processor configured to execute processor-executable routines and a tangible memory medium storing processor executable routines. The processor-executable routines, when executed by the processor, cause actions to be performed comprising: optionally clustering a plurality of reference sequences to produce a plurality of clusters; designing a plurality of tiled probes based on the clusters or on another subset of total reference sequences that has been selected using another approach such as longest reference sequence per taxonomic identifier, wherein a desired probe length and gap length between probes is specified; clustering the plurality of tiled probes with a threshold identity (e.g., 75%, 85%, 90%, 95%, 99% or 100% identity); optionally replacing ambiguous bases within the tiled probes based on a threshold tolerance; mapping the probes to all reference sequences and gap-filling based on a mismatch tolerance (mapping and gap-filling can be iterated up to n rounds, where n=the total # of available reference sequences per virus); optionally scoring the plurality of probes and predicting problematic probes; optionally performing one or both of removing problematic probe sequences or adding backup probes to supplement problematic probes; optionally further predicting difficult to synthesize probes; optionally replacing difficult to synthesize probes with a mutated probe; and optionally replacing ambiguous bases in the backup and/or mutated probes based on the threshold tolerance or a different threshold tolerance.

Additionally described herein are tangible computer-readable media encoding processor-executable routines, wherein the processor-executable routines comprise: code for optionally clustering a plurality of reference sequences to produce a plurality of clusters; code for designing a plurality of tiled probes based on the clusters or on another subset of total reference sequences that has been selected using another approach such as longest reference sequence per taxonomic identifier, wherein a desired probe length and gap length between probes is specified; code for clustering the plurality of tiled probes with a threshold identity (e.g., 75%, 85%, 90%, 95%, 99% or 100% identity); code for optionally replacing ambiguous bases within the tiled probes based on a threshold tolerance; code for mapping the probes to all reference sequences and gap-filling based on a mismatch tolerance (mapping and gap-filling can be iterated up to n rounds, where n=the total # of available reference sequences per virus); code for optionally scoring the plurality of probes and predicting problematic probes; code for optionally performing one or both of removing problematic probe sequences or adding backup probes to supplement problematic probes; code for optionally further predicting difficult to synthesize probes; code for optionally replacing difficult to synthesize probes with a mutated probe; and code for optionally replacing ambiguous bases in the backup and/or mutated probes based on the threshold tolerance or a different threshold tolerance.

In some embodiments, the reference sequence clustering step (a) is carried out a threshold identity (e.g., 75%, 85%, 90%, 95%, 99% or 100% identity). In some embodiments, the reference sequence clustering step (a) is carried out with 90% identity. In some embodiments, the reference sequence clustering step (a) is carried out with 95% identity. In some embodiments, the reference sequence clustering step (a) is carried out with 96% identity. In some embodiments, the reference sequence clustering step (a) is carried out with 97% identity. In some embodiments, the reference sequence clustering step (a) is carried out with 98% identity. In some embodiments, the reference sequence clustering step (a) is carried out with 99% identity.

In some embodiments, the problematic probes are predicted using base metric rules.

In some embodiments, the problematic probes are predicted using GC content, homopolymer length, and/or Shannon entropy calculations.

In some embodiments, the method further comprise scoring the best back up probes to supplement problematic probes.

In some embodiments, a desired gap length (e.g., 0 bp, 50 bp, 80 bp, 100 bp, 150 bp, 200 bp, 500 bp, 1000 bp, and so forth) between probes (e.g., probes having a length of approximately 80-mer to approximately 120-mer) is specified.

In some embodiments, the probes have a 0%, 1%, 5%, 10%, 15%, 20%, 25%, or 30% mismatch tolerance. In some embodiments, the probes have a 0% mismatch tolerance. In some embodiments, the probes have a 1% mismatch tolerance. In some embodiments, the probes have a 5% mismatch tolerance. In some embodiments, the probes have a 10% mismatch tolerance. In some embodiments, the probes have a 15% mismatch tolerance. In some embodiments, the probes have a 20% mismatch tolerance. In some embodiments, the probes have a 25% mismatch tolerance. In some embodiments, the probes have a 30% mismatch tolerance.

In some embodiments, the ambiguous bases are replaced with 1% to 10% tolerance (e.g., a 5% tolerance). In some embodiments, the ambiguous bases are replaced with 1% tolerance. In some embodiments, the ambiguous bases are replaced with 2% tolerance. In some embodiments, the ambiguous bases are replaced with 3% tolerance. In some embodiments, the ambiguous bases are replaced with 4% tolerance. In some embodiments, the ambiguous bases are replaced with 10% tolerance.

In some embodiments, the problematic probe sequences are determined by cross hybridization, human homology checks, ribosomal operon homology checks, transposable element checks, and/or by identifying low-complexity regions of target sequences.

In some embodiments, the mutated probe has from 1 to 10 (e.g., 5) mutations.

In some embodiments, the method or system further comprise production of a pool of probes.

In some embodiments, the threshold identity for probe clustering comprises identity in the range of 75% to 100% (e.g., 75%, 85%, 90%, 95%, 99% or 100% identity).

In some embodiments, all possible probes per target are designed and scored based on, but not limited to, GC content, homopolymer length, and/or Shannon entropy calculations. In further aspects an optimal set of non-overlapping probes is selected using an interval scheduling technique

In some embodiments, probe overlap optimization is performed to reduce probe redundancy, wherein the process iterates over every unique group of overlapping probes (pairs and trios) until the collapse rate plateaus

Other methods of designing probe sequences are described in PCT/US23/76171, which is incorporated herein by reference in its entirety.

### Viral Targets

Public health officials need to be able to detect viral pathogens in a variety of environmental samples to detect disease outbreaks in a population and measure the intensity of disease outbreaks. Thus, this approach may be used to detect a variety of viral pathogens.

In some embodiments, at least one viral molecule and/or viral probe target is selected from Adeno-associated virus 2 (AAV2), Aichi virus 1 (AiV-A1), Alkhumra hemorrhagic fever virus (AHFV), Andes virus (ANDV), Anjozorobe virus (ANJV), Araucaria virus, Australian bat lyssavirus (ABLV), Bayou virus (BAYV), BK polyomavirus (BKPyV), Black Creek Canal virus (BCCV), Bombali virus (BOMV), Bourbon virus (BRBV), Bundibugyo virus (BDBV), Cache Valley virus (CVV), California encephalitis virus (CEV), Cedar virus (CedV), Chapare virus (CHAPV), Chikungunya virus (CHIKV), Choclo virus (CHOV), Colorado tick fever virus (CTFV), Crimean-Congo hemorrhagic fever virus (CCHFV), Crimean-Congo hemorrhagic fever virus 2 (CCHFV-2), Dengue virus (DENV), Dobrava-Belgrade virus (DOBV), Duvenhage virus (DUVV), Eastern equine encephalitis virus (EEEV), Ebola virus (EBOV), Enterovirus A, Enterovirus B, Enterovirus C, Enterovirus D, Epstein-Barr virus (EBV), European bat lyssavirus (EBLV), Ghana virus (GhV), Guanarito virus (GTOV), Hantaan virus (HTNV), Heartland virus (HRTV), Hendra virus (HeV), Henipavirus unclassified, Hepatitis A virus (HAV), Hepatitis B virus (HBV), Hepatitis C virus (HCV), Hepatitis D virus (HDV), Hepatitis E virus (HEV), Herpes simplex virus 1 (HSV1), Herpes simplex virus 2 (HSV2), Human adenovirus A, Human adenovirus B, Human adenovirus C, Human adenovirus D, Human adenovirus E, Human adenovirus F, Human adenovirus G, Human bocavirus (HBOV), Human coronavirus 229E (HCOV_229E), Human coronavirus HKUI (HCOV_HKUI), Human coronavirus NL63 (HCOV_NL63), Human coronavirus OC43 (HCOV_OC43), Human cytomegalovirus (HCMV), Human immunodeficiency virus 1 (HIV-1), Human immunodeficiency virus 2 (HIV-2), Human metapneumovirus (HMPV), Human papillomavirus 11 (HPV11), Human papillomavirus 16 (HPV16; high-risk), Human papillomavirus 18 (HPV18; high-risk), Human papillomavirus 26 (HPV26), Human papillomavirus 31 (HPV31; high-risk), Human papillomavirus 33 (HPV33; high-risk), Human papillomavirus 35 (HPV35; high-risk), Human papillomavirus 39 (HPV39; high-risk), Human papillomavirus 40 (HPV40), Human papillomavirus 42 (HPV42), Human papillomavirus 43 (HPV43), Human papillomavirus 44 (HPV44), Human papillomavirus 45 (HPV45; high-risk), Human papillomavirus 51 (HPV51; high-risk), Human papillomavirus 52 (HPV52; high-risk), Human papillomavirus 53 (HPV53), Human papillomavirus 54 (HPV54), Human papillomavirus 56 (HPV56; high-risk), Human papillomavirus 58 (HPV58; high-risk), Human papillomavirus 59 (HPV59; high-risk), Human papillomavirus 6 (HPV6), Human papillomavirus 61 (HPV61), Human papillomavirus 66 (HPV66; high-risk), Human papillomavirus 68 (HPV68; high-risk), Human papillomavirus 69 (HPV69), Human papillomavirus 70 (HPV70), Human papillomavirus 73 (HPV73), Human papillomavirus 82 (HPV82), Human parainfluenza virus 1 (HPIV-1), Human parainfluenza virus 2 (HPIV-2), Human parainfluenza virus 3 (HPIV-3), Human parainfluenza virus 4 (HPIV-4), Human parechovirus (HPeV), Human parvovirus B19 (B19V), Human polyomavirus 6 (HPyV6), Human polyomavirus 7 (HPyV7), Human polyomavirus 9 (HPyV9), Human respiratory syncytial virus A (HRSV-A), Human respiratory syncytial virus B (HRSV-B), Influenza A virus, Influenza B virus, Influenza C virus, Isla Vista virus, Itapua virus, Jamestown Canyon virus (JCV), Japanese encephalitis virus (JEV), JC polyomavirus (JCPyV), Junin virus (JUNV), Juquitiba virus, KI polyomavirus (KIPyV), Kyasanur Forest disease virus (KFDV), La Crosse virus (LACV), Lagos bat virus (LBV), Laguna Negra virus (LANV), Langya virus, Lassa virus (LASV), LI polyomavirus (LIPyV), Lloviu virus (LLOV), Lujo virus (LUJV), Luxi virus (LUXV), Lymphocytic choriomeningitis virus (LCMV), Machupo virus (MACV), Mamastrovirus 1 (MAstV1), Mamastrovirus 6 (MAstV6), Mamastrovirus 8 (MAstV8), Mamastrovirus 9 (MAstV9), Maporal virus (MAPV), Marburg virus (MARV), Mayaro virus (MAYV), Measles virus (MV), Menangle virus (MenV), Merkel cell polyomavirus (MCPyV), Middle East respiratory syndrome-related coronavirus (MERS-COV), Mojiang virus (MojV), Mokola virus (MOKV), Monkeypox virus (MPV), Monongahela hantavirus, Muleshoe virus, Mumps virus (MuV), Murray Valley encephalitis virus (MVEV), MW polyomavirus (MWPyV), New Jersey polyomavirus (NJPyV), Nipah virus (NiV), Norovirus, Omsk hemorrhagic fever virus (OHFV), Onyong-nyong virus (ONNV), Oropouche virus (OROV), Paranoa virus, Powassan virus (POWV), Punta Toro virus (PTV), Puumala virus (PUUV), Rabies virus (RABV), Ravn virus (RAVV), Reston virus (RESTV), Rhinovirus A (RV-A), Rhinovirus B (RV-B), Rhinovirus C (RV-C), Rift Valley fever virus (RVFV), Ross River virus (RRV), Rotavirus A (RVA), Rotavirus B (RVB), Rotavirus C (RVC), Rubella virus (RuV), Sabia virus (SBAV), Salivirus A (SaV-A), Sandfly fever Sicilian virus (SFCV), Sangassou virus (SANGV), Sapovirus, Semliki Forest virus (SFV), Seoul virus (SEOV), Severe acute respiratory syndrome coronavirus (SARS-COV), Severe acute respiratory syndrome coronavirus 2 (SARS-CoV-2), Severe fever with thrombocytopenia syndrome virus (SFTSV), Simian virus 40 (SV40), Sin nombre virus (SNV), Sindbis virus (SINV), Snowshoe hare virus (SSHV), Sosuga virus (SoRV), St. Louis encephalitis virus (SLEV), STL polyomavirus (STLPyV), Sudan virus (SUDV), Tacheng tick virus 2 (TcTV-2), Tahyna virus (TAHV), Tai Forest virus (TAFV), Tick-borne encephalitis virus (TBEV), Torque teno virus (TTV), Toscana virus (TOSV), Trichodysplasia spinulosa-associated polyomavirus (TSPyV), Tula virus (TULV), Usutu virus (USUV), Varicella-zoster virus (VZV), Variola virus (VARV), Venezuelan equine encephalitis virus (VEEV), West Nile virus (WNV), Western equine encephalitis virus (WEEV), WU polyomavirus (WUPyV), Yellow fever virus (YFV), and Zika virus (ZIKV).

As used herein, the term “nucleic acid” is intended to be consistent with its use in the art and includes naturally occurring nucleic acids or functional analogs thereof. Particularly useful functional analogs are capable of hybridizing to a nucleic acid in a sequence specific fashion or capable of being used as a template for replication of a particular nucleotide sequence. Naturally occurring nucleic acids generally have a backbone containing phosphodiester bonds. An analog structure can have an alternate backbone linkage including any of a variety of those known in the art. Naturally occurring nucleic acids generally have a deoxyribose sugar (e.g., found in deoxyribonucleic acid (DNA)) or a ribose sugar (e.g., found in ribonucleic acid (RNA)). A nucleic acid can contain any of a variety of analogs of these sugar moieties that are known in the art. A nucleic acid can include native or non-native bases. In this regard, a native deoxyribonucleic acid can have one or more bases selected from the group consisting of adenine, thymine, cytosine or guanine and a ribonucleic acid can have one or more bases selected from the group consisting of uracil, adenine, cytosine, or guanine. Useful non-native bases that can be included in a nucleic acid are known in the art. The term “target,” when used in reference to a nucleic acid, is intended as a semantic identifier for the nucleic acid in the context of a method or composition set forth herein and does not necessarily limit the structure or function of the nucleic acid beyond what is otherwise explicitly indicated.

As used herein, “desired RNA” or “a desired RNA sequence” refers to any RNA that a user wants to analyze. As used herein, a desired RNA includes the complement of a desired RNA sequence. Desired RNA may be RNA from which a user would like to collect sequencing data, after cDNA and library preparation. In some instances, the desired RNA is mRNA (or messenger RNA). In some instances, the desired RNA is a portion of the mRNA in a sample.

As used herein, “desired library fragments” refers to library fragments prepared from cDNA prepared from desired RNA.

In some embodiments, the desired RNA sequence is sequence from a virus listed above.

### Example 1. Preparation of Probes to Improve Enrichment of Viruses of Interest in Samples

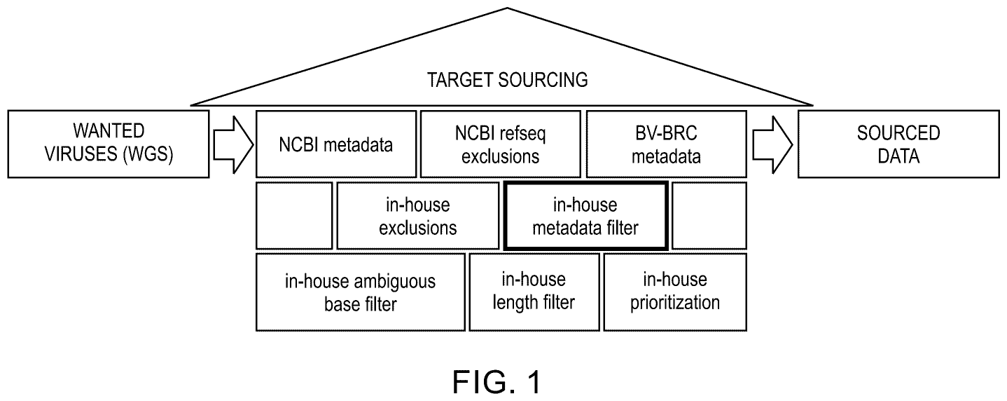

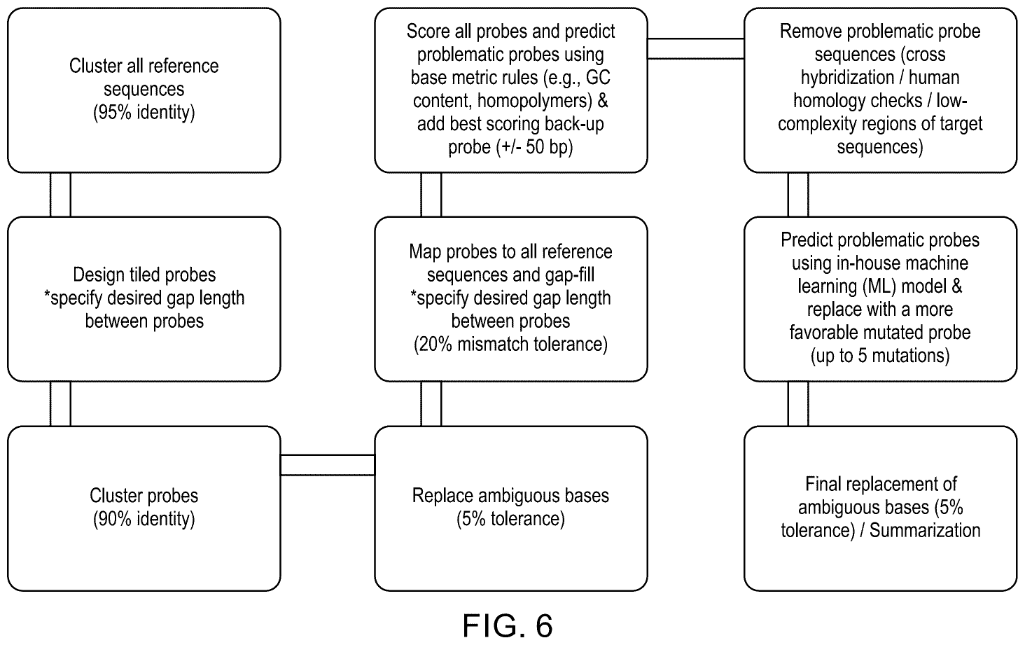

Viral reference sequences were sourced and viral probes designed as depicted in FIG. 1 and FIG. 6.

### Example 2. Probe Count Reduction

### A. Norovirus—Moderate Viral Genome Diversity Example

Using the methods from Example 1, 3,026 non-redundant norovirus reference sequences were sourced for viral WGS probe design. After 95% identity clustering, 234 representative norovirus reference sequences were obtained.

Using an iterative reference selection and gap-filling approach reduced probe count by more than 50%, as shown in Table 1. In this example, a set of Refseq reference genome sequences, each from a different human norovirus genotype, was selected for Round 1 probe design.

TABLE 1
Novovirus iterative reference selection

| | Round 1 reference | |
|---|---|---|
| Viral WGS design strategy | sequence selection | Probe count |
| No iterative design | NONE | 14,228 |
| Iterative | 14 Refseq reference | 6,689 |
| | genome sequences | |

### B. HIV-1—High Viral Genome Diversity Example

Using the methods from Example 1, 23,734 non-redundant HIV-1 reference sequences were sourced for viral WGS probe design. After 95% identity clustering, 15,318 representative norovirus reference sequences were obtained.

Using an iterative reference selection and gap-filling approach reduced probe count by more than 90%, as shown in Table 2. In this example, the optimal Round 1 reference selection identified was a set of reference genome sequences representing the longest sequence from all HIV-1 taxonomic identifiers (taxids).

TABLE 2
HIV-1 iterative reference selection

| Viral WGS | | Probe |
|---|---|---|
| design strategy | Round 1 reference sequence selection | count |
| Iterative | 4,019 reference sequences (90% clustered) | 156,880 |
| Iterative | 1,294 reference sequences (85% clustered) | 84,565 |
| Iterative | 508 reference sequences (80% clustered) | 63,533 |
| Iterative | 2,665 reference sequences (1 per taxid, | 12,454 |
| | longest available) | |

While the preceding describes certain aspects of the presently contemplated techniques, further implementations and details are contemplated and encompassed by the present disclosure. For example, improved prediction algorithms may be employed as part of the Round 1 reference sequence selection used in iterative viral probe design. Further, iterative viral probe design may be employed with exactly n rounds, (where n is the total number of reference sequences available), including optimization of the reference sequence order. Additionally, viral probe design methods may be employed where probes are designed on all available reference sequences, with the probes being subsequently ranked. Probe ranking in such implementations may prioritize probes that are designed across multiple reference sequences. In addition, viral probe design methods may be employed where available reference sequences are first aligned. In such an approach, available reference sequences may be optionally trimmed to have consistent boundaries. As may be appreciated, these various approaches and embodiments may be combined or all employed.

Similarly, the iterative viral probe design methods, systems, and any of these described process (e.g., algorithmic) variations may also be used for non-viral probe design (e.g., genomes or targeted regions from bacteria, fungi, parasites, and/or antimicrobial resistance markers).

It is to be understood that the subject matter described herein is not limited in its application to the details of construction and the arrangement of components set forth in the description herein or illustrated in the drawings hereof. The subject matter described herein is capable of other implementations and of being practiced or of being carried out in various ways. Also, it is to be understood that the phraseology and terminology used herein is for the purpose of description and should not be regarded as limiting. As used herein, an element or step recited in the singular and proceeded with the word “a” or “an” should be understood as not excluding plural of said elements or steps, unless such exclusion is explicitly stated. Furthermore, references to “one example” are not intended to be interpreted as excluding the existence of additional examples that also incorporate the recited features. The use of “including,” “comprising,” or “having” and variations thereof herein is meant to encompass the items listed thereafter and equivalents thereof as well as additional items.

When used in the claims, the term “set” should be understood as one or more things which are grouped together. Similarly, when used in the claims “based on” should be understood as indicating that one thing is determined at least in part by what it is specified as being “based on.”

It is to be understood that the above description is intended to be illustrative, and not restrictive. For example, the above-described examples (and/or aspects thereof) may be used in combination with each other. In addition, many modifications may be made to adapt a particular situation or material to the teachings of the presently described subject matter without departing from its scope. While the dimensions, types of materials and coatings described herein are intended to define the parameters of the disclosed subject matter, they are by no means limiting and instead illustrations. Many further examples will be apparent to those of skill in the art upon reviewing the above description. The scope of the disclosed subject matter should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. § 112(f) paragraph, unless and until such claim limitations expressly use the phrase “means for” followed by a statement of function void of further structure.

The following claims recite aspects of certain examples of the disclosed subject matter and are considered to be part of the above disclosure. These aspects may be combined with one another.

What is claimed is:

1. An iterative method of designing a pool of probes for enriching a sample for one or more target viral nucleic acids comprising the steps of:
clustering a plurality of reference sequences to produce a plurality of clusters;
designing a plurality of tiled probes based on the clusters, wherein a desired probe length and gap length between probes is specified;
clustering the plurality of tiled probes with a threshold identity;
replacing ambiguous bases within the tiled probes based on a threshold tolerance;
mapping the probes to all reference sequences and gap-filling based on a mismatch tolerance;
scoring the plurality of probes and predicting problematic probes;
performing one or both of removing problematic probe sequences or adding backup probes to supplement problematic probes;
predicting difficult to synthesize probes;
replacing difficult to synthesize probes with a mutated probe; and
replacing ambiguous bases in the backup and/or replacement probes based on the threshold tolerance or a different tolerance threshold.

2. The method of claim 1, wherein problematic probes are predicted using GC content, homopolymer length, and/or Shannon entropy calculations.

3. The method of claim 1, further comprising scoring back up probes to supplement problematic probes.

4. The method of claim 1, wherein a desired gap length between probes is specified.

5. The method of claim 1, wherein ambiguous bases are replaced with a 1% to 10% tolerance.

6. The method of claim 1, wherein the problematic probe sequences are determined by cross hybridization, human homology checks, ribosomal operon homology checks, transposable element checks, and/or by identifying low-complexity regions of target sequences.

7. The method of claim 1, wherein the mutated probe has from 1 to 10 mutations.

8. The method of claim 1, further comprising producing the pool of probes.

9. The method of claim 1, wherein possible probes per target are designed and scored based on one or more of GC content, homopolymer length, and/or Shannon entropy calculations.

10. The method of claim 1, wherein probe overlap optimization is performed to reduce probe redundancy.

11. A probe design system, comprising:
a processor configured to execute processor-executable routines;
a tangible memory medium storing processor executable routines, wherein the processor-executable routines, when executed by the processor, cause actions to be performed comprising:
clustering a plurality of reference sequences to produce a plurality of clusters;
designing a plurality of tiled probes based on the clusters, wherein a desired probe length and gap length between probes is specified;
clustering the plurality of tiled probes with a threshold identity;
replacing ambiguous bases within the tiled probes based on a threshold tolerance;
mapping the probes to all reference sequences and gap-filling based on a mismatch tolerance;
scoring the plurality of probes and predicting problematic probes;
performing one or both of removing problematic probe sequences or adding backup probes to supplement problematic probes;
predicting difficult to synthesize probes;
replacing difficult to synthesize probes with a mutated probe; and
replacing ambiguous bases in the backup and/or replacement probes based on the threshold tolerance or a different tolerance threshold.

12. The probe design system of claim 11, wherein the processor-executable routines, when executed by the processor, cause actions to be performed comprising: scoring back up probes to supplement problematic probes.

13. The probe design system of claim 11, wherein the problematic probe sequences are determined by cross hybridization, human homology checks, ribosomal operon homology checks, transposable element checks, and/or by identifying low-complexity regions of target sequences.

14. The probe design system of claim 11, wherein the mutated probe has from 1 to 10 mutations.

15. The probe design system of claim 11, wherein the processor-executable routines, when executed by the processor, cause actions to be performed comprising causing production of the pool of probes.

16. One or more tangible computer-readable media encoding processor-executable routines, wherein the processor-executable routines comprise:
code for clustering a plurality of reference sequences to produce a plurality of clusters;
code for designing a plurality of tiled probes based on the clusters, wherein a desired probe length and gap length between probes is specified;
code for clustering the plurality of tiled probes with a threshold identity;
code for replacing ambiguous bases within the tiled probes based on a threshold tolerance;
code for mapping the probes to all reference sequences and gap-filling based on a mismatch tolerance;
code for scoring the plurality of probes and predicting problematic probes;
code for performing one or both of removing problematic probe sequences or adding backup probes to supplement problematic probes;
code for predicting difficult to synthesize probes;
code for replacing difficult to synthesize probes with a mutated probe; and
code for replacing ambiguous bases in the backup and/or replacement probes based on the threshold tolerance or a different tolerance threshold.

17. The one or more tangible computer-readable media of claim 16, wherein the processor-executable routines, when executed by the processor, cause actions to be performed comprising: scoring back up probes to supplement problematic probes.

18. The one or more tangible computer-readable media of claim 16, wherein the problematic probe sequences are determined by cross hybridization, human homology checks, ribosomal operon homology checks, transposable element checks, and/or by identifying low-complexity regions of target sequences.

19. The one or more tangible computer-readable media of claim 16, wherein the mutated probe has from 1 to 10 mutations.

20. The one or more tangible computer-readable media of claim 16, wherein the processor-executable routines, when executed by the processor, cause actions to be performed comprising causing production of the pool of probes.
